(19) United States
(12) Patent Application Publication
(10) Pub. No.: US 2011/0092716 A1
(43) Pub. Date: Apr. 21, 2011

(54) DEHYDROHALOGENATION PROCESS FOR THE PREPARATION OF INTERMEDIATES USEFUL IN PROVIDING 6,6-DIMETHYL-3-AZABICYCLO-[3.1.0]- HEXANE COMPOUNDS

(76) Inventors: Daw-long Kwok, Gillette, NJ (US); Hong-Chang Lee, Livingston, NJ (US); Ilia A. Zavialov, Princeton, NJ (US)

(21) Appl. No.: 12/745,153

(22) PCT Filed: Nov. 20, 2008
(86) PCT No.: PCT/US08/84174

§ 371 Date: Dec. 8, 2010

Related U.S. Application Data
(60) Provisional application No. 61/004,601, filed on Nov. 28, 2007.

(51) Int. Cl. C07D 209/02

(52) U.S. Cl. 548/452

(57) ABSTRACT

The present invention provides for a process for preparing the pyrrole compounds of Formula Va and Vb.

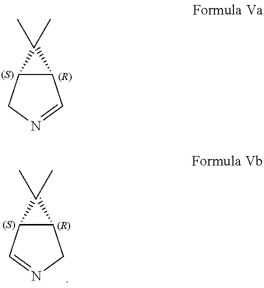

### CROSS REFERENCE TO RELATED APPLICATIONS

The present application is based on and claims the priority of U.S. Provisional Application 61/004,601 filed Nov. 28, 2007, which application is incorporated herein by reference in its entirety.

### FIELD OF THE INVENTION

The present invention relates to a process for preparing 6,6-Dimethyl-3-aza-bicyclo[3.1.0]hex-2-ene, useful as an intermediate in the preparation of compounds having activity as hepatitis C virus protease inhibitors.

### BACKGROUND OF THE INVENTION

Identification of any publication in this section or any section of this application is not an admission that such publication is prior art to the present invention.

The compound of Formula I, N-[3-amino-1-(cyclobutylmethyl)-2,3-dioxopropyl]-3-{N-[(tert-butylamino)carbonyl]-3-methyl-L-valyl}-6,6-dimethyl-3-azabicyclo[3.1.0]hexane-2-carboxamide, is useful for treating hepatitis C and related disorders. Specifically, the compound of Formula I is an inhibitor of the HCV NS3/NS4a serine protease.

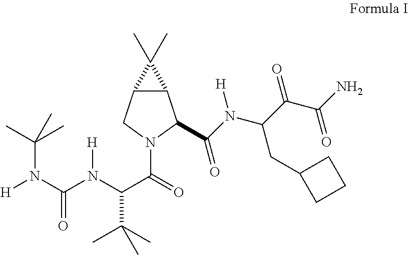

A process for making the compound of Formula I, (1R,2S,5S)—N-[(1S)-3-amino-1-(cyclobutylmethyl)-2,3-dioxopropyl]-3-[(2S)-2-[[[(1,1-dimethylethyl)amino]carbonyl]-amino]-3,3-dimethyl-1-oxobutyl]-6,6-dimethyl-3-azabicyclo[3.1.0]hexane-2-carboxamide is described in U.S. Pat. No. 7,012,066 (the '066 patent), Example XXIV, beginning at Column 448 therein. Additional processes for the preparation of the compounds of Formula I are described in published U.S. patent application nos. 2005/0249702, published Nov. 10, 2005, and 2005/0059800, published Mar. 17, 2005, and in Provisional application Nos. 60/876,296 and 60/876,447, each of which was filed Dec. 20, 2006, each of which applications are incorporated herein by reference in their entirety.

In general, the process for the preparation of compounds of Formula I is illustrated in Scheme I:

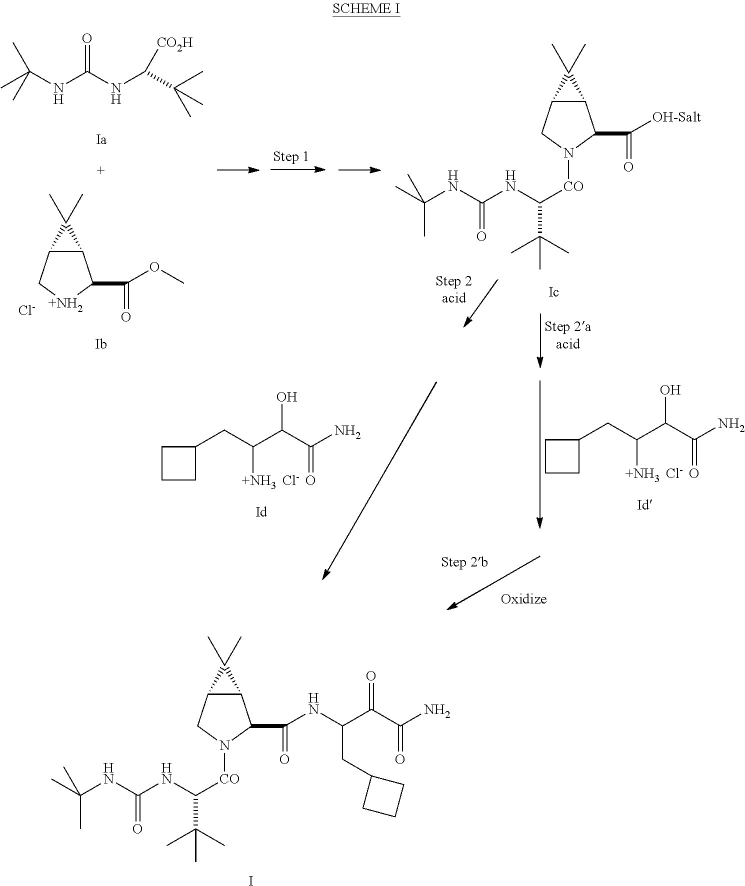

As can be seen from the foregoing, esters of 6,6-dimethyl-3-azabicyclo[3.1.0]hexane-2-carboxylic acid, or a salt thereof, for example, the salt compound of Formula Ib shown in Scheme I above, are useful as intermediates in the synthesis of HCV protease inhibiting compounds. The salt compound of Formula Ib, (1R,2S,5S)-6,6-dimethyl-3-azabicyclo[3.1.0]-hexane-2-carboxylic acid, methyl ester hydrochloride, disclosed in US Publication No. 2003-0216325 A1 which is incorporated herein by reference, is a key intermediate used in preparation of the hepatitis C virus (“HCV”) protease inhibitor having the following structure of Formula I, discussed above.

Various methods are known in the art to make esters of 6,6-dimethyl-3-azabicyclo[3.1.0]hexane-2-carboxylic acid, for example, US Publication No. 2003-0216325 A1 discloses preparation of compound 1.

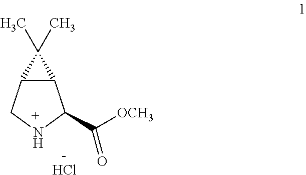

from the corresponding alcohol 2

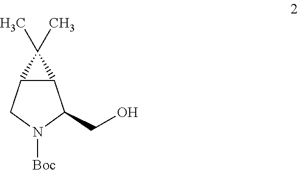

by performing a Jones oxidation and then cleaving the protection with methanolic HCl. This procedure modifies the one disclosed by R. Zhang and J. S. Madalengoitia in J. Org. Chem., 64, pp 330-31 (1999).

US Publication No. US 2005/0020689 A1, herein incorporated by reference, discloses a process for making 3-(amino)-3-cyclobutyl methyl-2-hydroxy-propionamide or a salt thereof, which is an intermediate in the synthesis of compound Z. This publication also claims some intermediates prepared in the synthesis.

US Publication No. US 2005/0059800, herein incorporated by reference, claims an alternative process for preparing the compound of formula Z, which involves using methyl 6,6-dimethyl-3-azabicyclo[3.1.0]hexane-2-carboxylic acid as a starting material.

US Publication No. US 2005/0059684 A1, herein incorporated by reference, prepares esters of 6,6-dimethyl-3-azabicyclo[3.1.0]hexane-2-carboxylic acid in a process summarized by Scheme II.

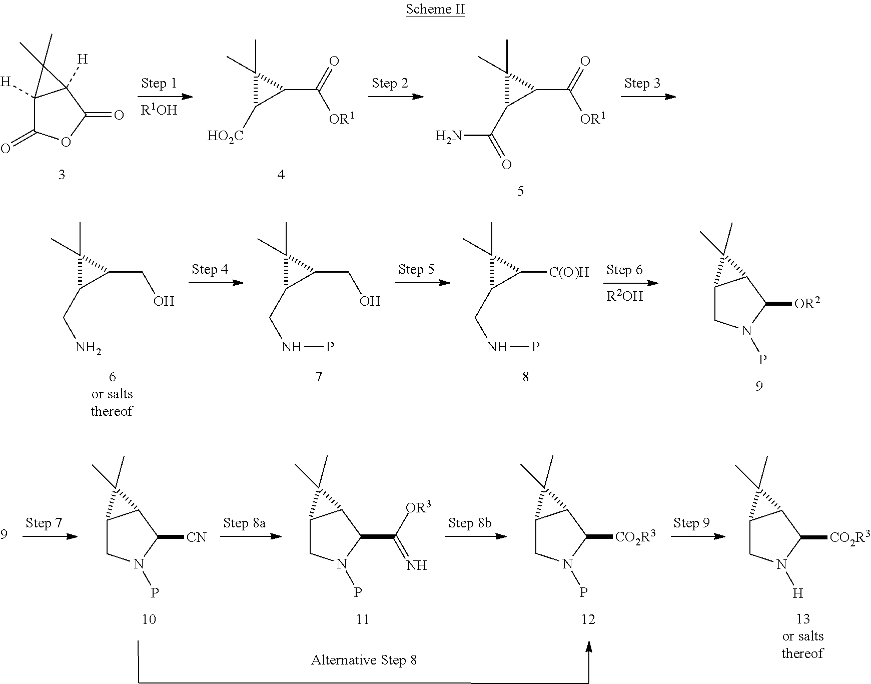

EP 0 010 799 (the '799 publication) discloses a process for preparing acid compounds of the formula

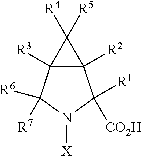

where R¹ is hydrogen or alkyl and R² to R⁷ are, for example, alkyl, from the corresponding imine through a nitrile intermediate. Accordingly, the imine is reacted with a cyanating reagent to form the corresponding nitrile, which is subsequently hydrolyzed to form the acid derivative. The imine derivative is prepared by direct oxidation of a bicyclo-pyrrolidine compound of the formula

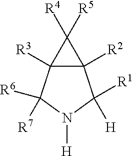

or by dehydrohalogenation of the corresponding halo-pyrrolidine derivative of the bicycle-pyrrolidine. The document indicates that the cyanation step forming the nitrile generally leads exclusively to the formation of the trans geometric isomer and this stereochemistry is retained in the hydrolysis step.

U.S. Pat. No. 4,691,022 discloses a process for preparing an acid or ester derivatives of the formula

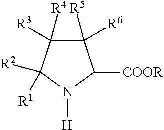

where R is hydrogen or alkyl and R⁴ and R⁵, for example, may form a bicyclic ring system, from the corresponding nitrile. The process comprises converting, with an oxidizing agent in the presence of a silver salt, a pyrrolidine derivative into the corresponding Δ¹-pyrrolidine derivative and subsequently reacting the pyrrolidine derivative with HCN, preferably generated by adding a metal cyanide in the presence of mineral acid to the reaction mixture, to form the nitrile. The product is prepared by subjecting the resulting nitrile to solvolysis. The patent does not disclose a process for making a particular isomer of these compounds in a high enantiomeric excess.

These foregoing processes comprise arduous reaction schemes and provide a statistical mixture of all possible isomers of the product bicyclo-pyrrolidine compound. There remains a need for methods amenable to commercial scale process for providing intermediates useful in the synthesis of compounds useful in the treatment or prevention or amelioration of one or more symptoms of hepatitis C. Further, there remains a need for processes providing enantiomeric intermediate products which provide prevalently the desired enantiomer without requiring arduous enantiomer separation techniques, for example, chiral chromatography.

U.S. Provisional Application Ser. Nos. 60/876,447 (the '447 application) and 60/876,296 (the '296 application), each filed Dec. 20, 2006, and International Applications filed herewith under Attorney docket numbers CD06582 (the '6582 application) and CD06583 (the '6583 application), all four of which are incorporated herein by reference in their entirety, disclose, in accordance with Scheme III, the preparation of a racemic mixture of esters of 6,6-dimethyl-3-azabicyclo[3.1.0]hexane-2-carboxylic acid esters (VIIa and VIIb) via 6,6-Dimethyl-3-aza-bicyclo[3.1.0]hexane (IV) prepared from caronic anhydride.

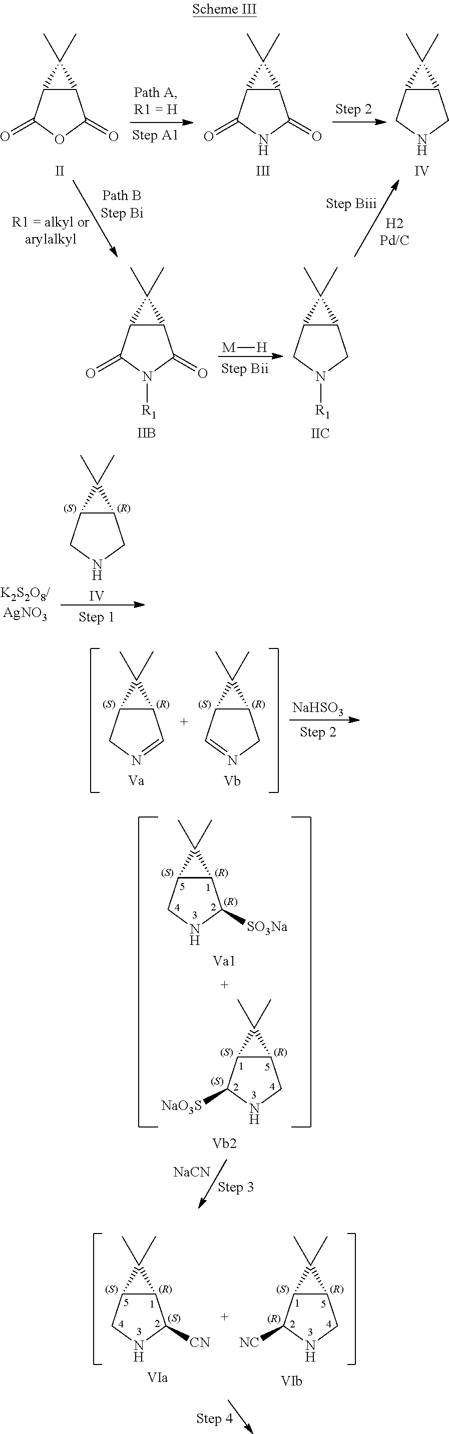

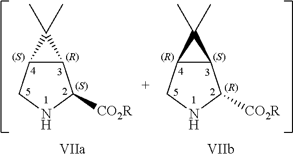

The processes described in each of the '447, '296, '6482, and '6483 applications, summarized in Scheme 2, above, provide the desired intermediate compound of Formula VIIa in a form which can be resolved from the isomeric compound of Formula VIIb by precipitating the desired intermediate from a racemate solution as the salt of a chiral acid. Each of these processes utilizes an imine intermediate, for example, the compounds of Formulae (Va) and (Vb). In each of these processes, the imine intermediate is provided by oxidizing the corresponding amine, for example, 6,6-Dimethyl-3-aza-bicyclo[3.1.0]hexane (IV), using utilizing potassium peroxodisulfate in the presence of a catalytic amount of silver nitrate. This process typically provides a racemic mixture of the imine in an overall yield of less than about 80%, which translates to a yield of less than about 40% of the desired imine isomer from the starting bicyclo-amine, for example, the compound of Formula (IV). This process also requires careful attention to various process variables, for example, temperature and agitation rate, to insure that the process consistently provides the imine product at the desired yields. Moreover, potassium peroxodisulfate is a strong oxidizer which must be handled in solution with the oxidation substrate introduced into the oxidizer solution to minimize loss of substrate in the process. Additionally, the oxidation reaction must be run at sub-ambient temperature and requires an extended reaction period to complete oxidation of the substrate. Moreover, the process requires workup of the reaction mixture by extraction of the reaction mixture with tert.-butylmethylether and fractional distillation to yield the product.

In view of the importance of imine intermediates of Formula Va in the synthesis of hepatitis C virus (“HCV”) protease inhibitors, novel methods for improving the efficiency and safety aspects of their provision are of interest.

### SUMMARY OF THE INVENTION

These and other needs are met by the present invention, which in one aspect provides a process for the provision of a mixture of compounds of the formulae (Va) and (Vb).

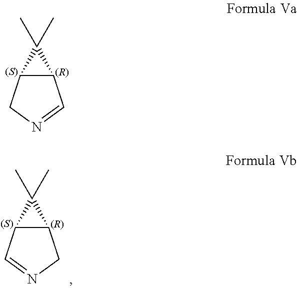

the process comprising:
(a) Treating a pyrrolidine compound of Formula IV,

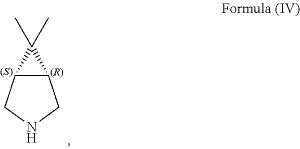

with a chlorinating agent to form the compound of Formula IVa,

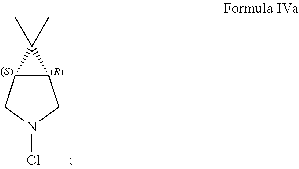

and
(b) dehydrochlorinating the chloroamine obtained in Step “a” to produce the compounds of Formulae VIa and VIb.

In some embodiments of the invention it is preferred to provide the compound of Formula IV by reacting caronic anhydride benzylamine, to form the aza-bicylcohexane dione compound (benzyl imide) of Formula IIB,

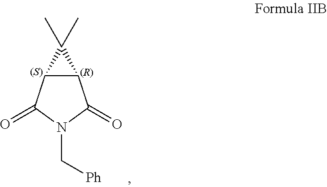

followed by reducing in turn both of the carbonyl functional groups of the benzyl imide, yielding an N-benzyl pyrrolidine compound of the Formula IIIc

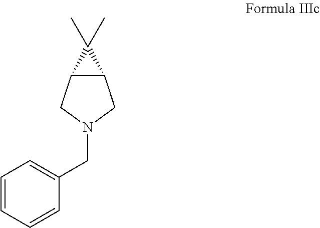

which is in turn reduced to a pyrrolidine (with elimination of the benzyl moiety). In some embodiments, it is preferred to reduce the ketone functional groups of the benzyl imide compound of Formula IIB by treating the compound with a metal hydride reagent, preferably lithium aluminum hydride, yielding the compound of Formula IIC. In some embodiments it is preferred to reduce the benzyl pyrrolidine compound of Formula IIIc using a group 8 metal catalyst and hydrogen, preferably the catalyst is palladium on carbon.

In some embodiments it is preferred to form the chloroamine of Formula IVa in a chlorination reaction wherein the chlorinating reagent selected from sodium hypochlorite and N-chlorosuccinamide (NCS) in an appropriate solvent. In some embodiments it is preferred to carry out the chlorination reaction in methyl-tertiarybutyl-ether (MTBE) as the chlorination solvent.

In some embodiments it is preferred to carry out the conversion of the chloroamine compound of Formula IVa to an imine by treating the compound of Formula IVa with a metal hydroxide, for example, potassium hydroxide and sodium hydroxide. In some embodiments it is preferred to carry out dehydrochlorination using a lipophilic phase transfer catalyst, for example, tetrabutyl ammonium hydroxide and N-benzyl cinchonidinium chloride. In some embodiments utilizing a phase transfer catalyst it is preferred to include a promoter in the reaction mixture, for example, short carbon chain alkanols, for example, methanol, ethanol, and isopropanol.

Other aspects of the invention will become apparent from the following detailed description.

### DESCRIPTION OF THE INVENTION

As used above, and throughout the specification, the following terms, unless otherwise indicated, shall be understood to have the following meanings:

“Alkyl” means an aliphatic hydrocarbon group which may be straight or branched, and optionally substituted at any position with one or more of any of the moieties listed below and comprising about 1 to about 20 carbon atoms in the chain. Preferred alkyl groups contain a carbon chain comprising about 1 to about 12 carbon atoms and may additional comprise appended thereto one or more substituents as defined above. More preferred alkyl groups contain a carbon chain comprising about 1 to about 6 carbon atoms and may additionally comprise appended thereto one or more substituents. Branched means that one or more lower alkyl groups such as methyl, ethyl or propyl, are attached as a substituent to a linear alkyl chain. “Lower alkyl” means a group having about 1 to about 6 carbon atoms in the chain which may be straight or branched and which may additionally comprise one or more substituents, as defined below, appended thereto. Non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl.

“Alkenyl” means an aliphatic hydrocarbon group containing at least one carbon-carbon double bond and which may be straight or branched and optionally comprise one or more substituents, as defined above. Preferably, an alkenyl moiety comprises from about 2 to about 15 carbon atoms, and may optionally contain one or more substituents, more preferably alkenyl groups have a chain comprising from about 2 to about 12 carbon atoms and may optionally additionally contain one or more substituents; and more preferably alkenyl groups have a chain comprising from about 2 to about 6 carbon atoms which may optionally have one or more substituents appended thereto.

The term “substituted alkenyl” means that the alkenyl group may have appended thereto one or more of the moieties listed herein, each of these substituents being independently selected from any of the moieties defined in this list, preferably the group consisting of halo, alkyl, aryl, cycloalkyl, cyano, and alkoxy. Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl, 3-methylbutynyl, n-pentynyl, and decynyl. The term “substituted alkynyl” means that the alkynyl group may be substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

“Aralkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting examples of suitable alkylaryl groups include o-tolyl, p-tolyl and xylyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more of the moieties defined in this list, each moiety being selected independently. Non-limiting examples of suitable monocyclic cycloalkyls include cyclopropyl, cyclopentyl, cyclohexyl, and cycloheptyl. Non-limiting examples of suitable multicyclic cycloalkyls include 1-decalin, norbornyl, and adamantyl moieties.

“Halo” means fluoro, chloro, bromo, and iodo groups. “Halogen” means fluorine, chlorine, bromine, or iodine. Preferred are fluorine, chlorine or bromine, and more preferred are fluorine and chlorine.

“Ring system substituent” means a substituent attached to an aromatic or non-aromatic ring system which, for example, replaces an available hydrogen on the ring system. Ring system substituents may be the same or different, each being independently selected from the group consisting of aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different and are independently selected from the group consisting of hydrogen, alkyl, aryl, and aralkyl.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cycloheptenyl, and the like. Non-limiting example of a suitable multicyclic cycloalkenyl is norbornylenyl.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable monocyclic azaheterocyclenyl groups include 1,2,3,4-tetrahydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Non-limiting examples of suitable oxaheterocyclenyl groups include 3,4-dihydro-2H-pyran, dihydrofuranyl, fluorodihydrofuranyl, and the like. Non-limiting example of a suitable multicyclic oxaheterocyclenyl group is 7-oxabicyclo[2.2.1]heptenyl. Non-limiting examples of suitable monocyclic thiaheterocyclenyl rings include dihydrothiophenyl, dihydrothiopyranyl, and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Aralkenyl” means an aryl-alkenyl-group in which the aryl and alkenyl are as previously described. Preferred aralkenyls contain a lower alkenyl group. Non-limiting examples of suitable aralkenyl groups include 2-phenethenyl and 2-naphthylethenyl. The bond to the parent moiety is through the alkenyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, 2-(furan-3-yl)ethyl and quinolin-3-ylmethyl. The bond to the parent moiety is through the alkyl.

“Heteroaralkenyl” means an heteroaryl-alkenyl-group in which the heteroaryl and alkenyl are as previously described. Preferred heteroaralkenyls contain a lower alkenyl group. Non-limiting examples of suitable heteroaralkenyl groups include 2-(pyrid-3-yl)ethenyl and 2-(quinolin-3-yl)ethenyl. The bond to the parent moiety is through the alkenyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an organic acid group in which the —OH of the carboxyl group is replaced by some other substituent, such as those defined above. Suitable, non-limiting examples include: H—C(O)—, alkyl-C(O)—, alkenyl-C(O)—, Alkynyl-C(O)—, cycloalkyl-C(O)—, cycloalkenyl-C(O)—, or cycloalkynyl-C(O)— group in which the various groups are as previously described. The bond to the parent moiety is through the carbonyl. Preferred acyls contain a lower alkyl. Non-limiting examples of suitable acyl groups include formyl, acetyl, propanoyl, 2-methylpropanoyl, butanoyl and cyclohexanoyl. The C═O moiety of the acyl group is a carboxyl moiety, and the term “carboxyl group” as used herein refers to —C(O)— moiety incorporated into a larger molecular fragment.

“Aroyl” means an aryl-C(O)— group in which the aryl group is as previously described. The bond to the parent moiety is through the carbonyl. Non-limiting examples of suitable groups include benzoyl and 1- and 2-naphthoyl.

“Alkoxy” means an alkyl-O— group in which the alkyl group is as previously described. Non-limiting examples of suitable alkoxy groups include methoxy, ethoxy, n-propoxy, isopropoxy, n-butoxy and heptoxy. The bond to the parent moiety is through the ether oxygen.

The term “optionally substituted” means optional substitution with the specified groups, radicals or moieties.

Enantiomeric excess (“e.e.”) is a percentage expressing the extent to which one enantiomer (e.g., R-enantiomer) is produced over the other (e.g. S-enantiomer), calculated by subtracting the difference in the amount of each enantiomer produced divided by the sum of the amount of each enantiomer produced

In one embodiment, the present invention provides a process for preparing a mixture of the compounds of Formulae Va and Vb. In some embodiments of the process it is preferred to provided the compounds in accordance with the process presented schematically in Scheme II, wherein caronic anhydride (II) is reacted to provide a pyrrolidine dione (IIB and III) which is in turn reduced to provide a pyrrolidine (IV and IIIB), from which a chloroamine is prepared ((IVa), which is in turn dehydrochlorinated to provide an imine (Va and Vb).

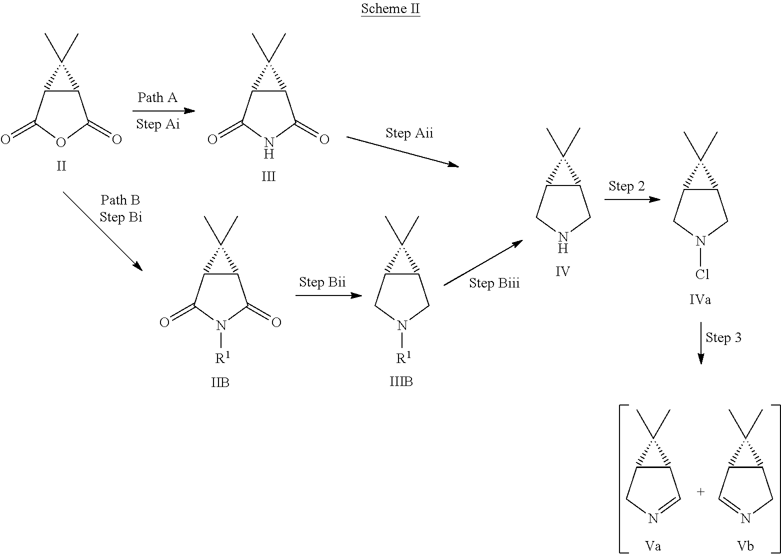

As used in Scheme II, R¹ is an aralkyl, substituted aralkyl or alkenyl (e.g., allyl) group, preferably a benzyl group.

There follows a detailed discussion of each of the steps of the process represented in Scheme II and intermediates prepared in each step.

### Step 1—Imide Formation

In some embodiments it is preferred to provide the desired imine from an aza-bicyclo(3.1.0)hexane compound of Formula IV, which my be obtained from an imide precursor of either Formula IIB or Formula III. The imide of either Formulae IIB or III may be prepared using caronic acid anhydride (H) starting material and one of the two procedures presented schematically in Scheme II identified as Path A and Path B. The process of Path A forms the imide of Formula III, which is reduced in one step to the precursor compound of Formula IV by treatment with a metal hydride, preferably lithium aluminum hydride.

The process of Path B forms the imide of Formula IIB by reacting caronic anhydride with benzyl amine, yielding the imide benzyl pyrrolidine dione (IIB). The benzyl pyrrolidine dione thus produced can be reduced two steps to the precursor compound of Formula IV. The carbonyl groups of the dione can be reduced with a metal hydride. The nitrogen moiety in the pyrrolidine ring can be hydrogenated with the elimination of the benzyl moiety by treatment of the pyrrolidine with hydrogen in the presence of a group 8 metal hydrogenation catalyst. Reduction of the carbonyl groups can be carried before or after hydrogenation of the nitrogen moiety. Accordingly, benzyl pyrrolidine (IIIB) can be provided from benzyl pyrrolidine dione (IIB), which is then hydrogenated to provide the pyrrolidine compound of Formula IV. Alternatively, the benzyl pyrrolidine dione (IIB) can be hydrogenated to provide pyrrolidine dione (III), which is then reduced with a metal hydride to provide the pyrrolidine compound of Formula (IV). Each of these process is illustrated next.

### Path A:

Caronic anhydride (formula II) can be catalytically converted, in a suitable solvent, to yield the imide of formula III. In some embodiments of the invention it is preferred to employ at least one solvent selected from water, tetrahydrofuran, methanol, isopropanol, methyl isobutyl ketone, xylenes, and formamide. Suitable catalysts for carrying out this conversion include, for example, 4-N,N-dimethylaminopyridine (DMAP) and lutidine. The catalyst is employed in the presence of a nitrogen source. Suitable nitrogen sources include, but are not limited to, NH₃, NH₄OH, H₂NC(O)NH₂, H₂NC(O)H, NH₄O₂CH, and NH₄O₂CCH₃. In some embodiments it is preferred to carry out the reaction at a temperature of from about 10° C. to about 200° C. After the imide of Formula III has been obtained, in Step Aii the carbonyl functional groups are reduced, preferably with a metal hydride, for example, LiAlH₄, yielding the compound of Formula IV. Preferably the reduction is carried out in ethereal solvents, for example, tetrahydrofuran (THF) and methyl-tertiarybutyl ether (MTBE).

### Path B:

A second method for the provision of the compound of Formula (IV) from caronic anhydride (Formula II) includes formation of an alkyl, allyl, or aralkyl imide (for example, the 6,6-Dimethyl-3-aza-bicyclo[3.1.0]hexane-2,4-dione imide compound of Formula IIB), which is then subjected to a two-step process comprising reduction of the dione and hydrogenation of the pyrrolidine nitrogen to yield the compound of Formula (IV). The two steps of this second method are described next.

Step Bi:

An intermediate imide of formula IIB is prepared from caronic anhydride by reaction with at least one reagent selected from an aralkylamine, substituted aralkylamine, and alkenylamine, in the presence of a solvent. In some embodiments of the invention it is preferred to employ amines selected from ArylCH₂NH₂ (benzyl amine) and AllylNH₂. In some embodiments of the invention it is preferred to use a solvent selected from t-butyl methylether (TBME), tetrahydrofuran, methanol, toluene, xylene and mixtures of two or more thereof. In some embodiments of the invention it is preferred to carry out the reaction at a temperature of from about 0° C. to about 200° C.

Step Bii:

The intermediate alkylimide of formula IIB prepared in Step Bi can be converted to compound IIIB by reducing the carbonyl groups in the imide ring, preferably using a metal hydride in an appropriate solvent. In some embodiments it is preferred to carry out this reduction using a reagent selected from lithium aluminum hydride (“LiAlH₄”), sodium bis(2-methoxyethoxy)aluminum dihydride (“Red-Al®”), and borane. In some embodiments of the invention it is preferred to carry out the reduction reaction in a solvent selected from tetrahydrofuran, 2-methyl tetrahydrofuran, tert-butyl methyl ether, 1,2-dimethoxyethane, toluene and mixtures of two or more thereof. In some embodiments it is preferred to isolate the product by distilling off the solvent. In some embodiments of the invention it is preferred to carry out the reduction reaction at a temperatures of from about −20° C. to about 80° C.

Step Biii:

Either before or after reduction of the imide in Step Bii, the nitrogen of the pyrrolidine ring is hydrogenated, with concomitant loss of the benzyl moiety, yielding the corresponding pyrrolidine compound. If reduction of the carbonyl moieties of the dione has been carried out, the hydrogenation yields the pyrrolidine of Formula IV (6,6-dimethyl-3-aza-bicyclo[3.1.0]hexane). If the dione reduction step is carried out after the hydrogenation step, hydrogenation yields the pyrrolidine dione (imide) of Formula III. In some embodiments it is preferred to carry out the hydrogenating step (Step Biii) using metal-mediated hydrogenolysis reaction conditions. In some embodiments it is preferred to use a catalyst comprising palladium on carbon (Pd/C) in the presence of hydrogen gas. One example of suitable reaction conditions can be found in the following reference: R. C. Bernotas and R. V. Cube, Synthetic Communication, 1990, 20, 1209.

Optionally, the compound of formula IV may be converted to the corresponding salt (compound of formula IVB) by reacting it with an acid. Suitable acids include, but are not limited to, mineral acids, for example, HCl, HBr, HI, HNO₃ or H₂SO₄. In some embodiments it is preferred to use a suitable organic solvent to provide a mineral acid solution for this treatment, for example, alcohol solvents, for example methanol and isopropanol.

Methods to prepare caronic anhydride are known in the art and this compound may be made, for example, from the synthesis disclosed in US Publication No. 2005/0059648 A1, which in Example 1 therein details a method for preparing the anhydride from ethyl chrysanthemumate in accordance with published procedures.

An alternate procedure, shown in Scheme IV, may be used to provide the starting material, which can be isolated, or used in situ to form the compound of formula III.

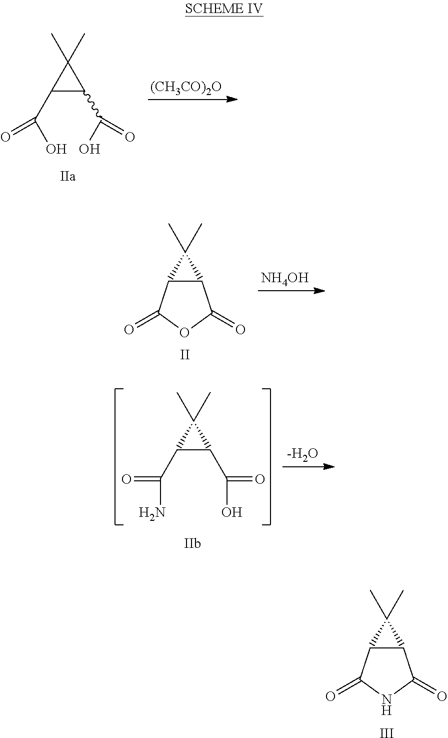

As shown in Scheme III, racemic 3,3 dimethyl-cyclopropane-1,2-dicarboxylic acid (IIa) is dissolved/suspended in toluene and treated with acetic anhydride in the presence of sulfuric acid to form cis-caronic anhydride preferentially (formula II). The cis-caronic anhydride may be isolated for use in a process in accordance with either Path A or Path B to provide an imide of Formula III or Formula IIB respectively, or it may be treated in situ with ammonium hydroxide forming the ring-opened intermediate, which is subsequently heated in situ to form, in a one-pot reaction, the imide of Formula III.

### Step 2 Chlorination of the Compound of Formula IV:

After the pyrrolidine of Formula IV has been prepared, it is converted to a chloroamine derivative (the compound of Formula IVa) by reaction with a chlorinating agent. In some embodiments it is preferred to select N-chlorosuccinamide (NCS) as the chlorinating reagent. In some embodiments it is preferred to select aqueous sodium hypochlorite as the chlorinating agent, preferably 13% wt. aqueous sodium hypochlorite. In some embodiments in which sodium hypochlorite or NCS is used as the chlorinating agent, the reaction is carried out in a low-polarity solvent, preferably an ether, preferably methyl-tert.-butyl ether (MTBE). In some embodiments in which the solvent is MTBE and the chlorinating agent is NCS, following the reaction the solid succininimide is removed from the reaction mixture by filtration and the resultant solution is concentrated prior to employing it in a dehydrochlorination step which yields the desired imine product. Optionally, the chloramine prepared in Step 2 can be isolated for later used in a dehydrochlorination reaction.

It will be appreciated that any means of providing the chloroamine of Formula IVa can be employed and still be within the scope of the present invention.

### Step 3 Dehydrochlorination to Yield the Imines of Formula Va and Vb:

The chloroamine of Formula IVa prepared in Step 2 is dehydrochlorinated to yield a corresponding imine of Formulae Va and Vb. Since the double bond introduced into the pyrrole ring can be introduced in either of two locations on the ring, this step yields a product mixture containing both Formulae Va and Vb isomers. In general, dehydrochlorination can be carried out by treating the compound of Formula IVa with a base. Non-limiting examples of suitable bases include, a metal hydroxide, for example potassium hydroxide and sodium hydroxide, a metal alkoxide, for example sodium and potassium alkoxide, and an amidine, for example 1,8-diazabicyclo[5.4.0]undec-7-ene (DBU).

In some embodiments, it is preferred to carry out the dehydrochlorination reaction by employing the base in an aqueous solvent mixed with a concentrate of the reaction mixture solvent in which the dehydrochlorination substrate (compound of Formula IVa) was prepared. In some embodiments it is preferred to dissolve the base in methanol or to include methanol in the base solvent.

In some embodiments, it is preferred to carry out the dehydrochlorination reaction in the presence of a lipophilic phase transfer catalyst, thus minimizing losses between the aqueous and organic phase, and minimizing the amount of solvent swapping involved in preparing and working up the reaction. Accordingly, in some embodiments it is preferred to employ a high lipophilic phase transfer catalyst (PTC), for example, tetraoctylammonium chloride, tetrabutylammonium chloride, and tetrabutyl ammonium hydroxide in the dehydrochlorination reaction. In some embodiments using a phase transfer catalyst, it is preferred to employ a cocatalyst, for example, short carbon chain alkyl alcohols, for example, methanol, ethanol, and isopropanol, along with the phase transfer catalyst to improve reaction rate and yield.

As mentioned above, the dehydrochlorination reaction can place the double-bond in the imine product in two different locations, resulting in the provision of a product containing a mixture of enantiomers. In some embodiments it is preferred to employ a chiral phase transfer catalyst in the reaction mixture to carry out the dehydrochlorination reaction, preferentially deprotonating the pyrrolidine to preferential yield the 1S, 5R pyrrole compound of Formula Va. Without wanting to be bound by theory, the inventors believe that the use of a chiral phase transfer catalyst will lead to the provision of an excess of one particular isomer. Examples of chiral phase transfer catalysts include (8S,9R)-(−)-N-benzyl cinchonidinium chloride.

It will be appreciated that similar yield improvements may be available from the process of the present invention carried out using other halogenating agents in place of chlorination reagents, thus, the process of the present invention can be carried out using halogenation/dehalogenation processes generally, for example, bromination/dehydrobromination or iodination/dehydroiodination, and still be within the scope of the present invention.

The following non-limiting EXAMPLES are provided to illustrate further the present invention. It will be apparent to those skilled in the art that many modifications, variations and alterations to the present disclosure, both to materials, methods and reaction conditions, may be practiced. All such modifications, variations, and alterations are intended to be within the spirit and scope of the present invention.

### EXAMPLES

Unless otherwise stated, all solvents and reagents are articles of commerce, and used as received. Unless otherwise stated, the following abbreviations have the stated meanings in the Examples below:

mL=milliliters

g=grams

eq=equivalents

THF=tetrahydrofuran

MeOH=methanol

Me=methyl

TBME=methyl tert-butyl ether

ACN=acetonitrile

Ph=phenyl

### Example I

### Preparation of the Compound of Formula IV

### Step 1: Preparation of 6,6-Dimethyl-3-aza-bicyclo[3.1.0]hexane-2,4-dione (III)

### Procedure A:

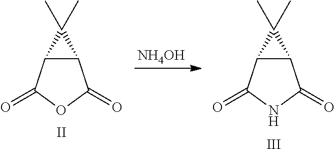

### Example A1

To a flask was charged 300 g of II (2.1 mol, 1 eq.) and 300 mL of water. While stirring, the mixture was cooled to 0 to 10° C. 225 mL of NH₄OH solution (14.8 M NH₃ in water) (3.3 mol, 1.5 eq.) were slowly added to the reaction mixture while stirring. During addition the reaction mixture temperature was maintained below 40° C. After the addition was complete, the batch was warmed to 105 to 115° C. and the water was collected by distillation while avoiding steam-distilling the product. Once the distillation was complete, the reaction mixture was heated gradually to between 165 to 180° C. to complete the cyclization. The reaction mixture was then cooled to a temperature between 60 to 70° C. and 200 mL of THF were added. The reaction mixture was reheated to 135 to 140° C. and the solvent was collected by distillation. The reaction mixture was recooled to a temperature between 60 and 70° C. and 200 mL of THF and 500 mL of n-heptane were added. The reaction mixture was cooled to 0 to 10° C. over a 5 hour period and then stirred for 0.5 to 1 hr and the product was crystallized. The crystals were collected, washed, and dried to yield compound III as a white crystalline powder (yield 90-95%). ¹H NMR (CDCl₃) δ 7.55 (bs, 1H), 2.31 (d, J=1.12 Hz, 2H), 1.35 (s, 3H), 1.24 (s, 3H).

### Example A2

Into a 12 L flask equipped with a temperature probe, distillation apparatus and mechanical stirrer was charged 1500.0 g of caronic anhydride (formula II, 10.7 mol). To the flask was added 1500 mL water followed by dropwise addition of NH₄OH (273.4 g, 16.1 mol). Water was collected by distillation at atmospheric for 2 h. The mixture was then heated to 155° C. and stirred an additional 22 hours. Analysis by ¹H NMR and HPLC indicated incomplete conversion to product. To the mixture was then added additional NH₄OH (50.4 g, 3.0 mol). The mixture was heated to 155° C. for 1 h. The reaction mixture was cooled to 120° C. and 7500 mL of normal butylacetate (n-BuOAc) was charged dropwise to the flask. The mixture was heated and maintained at a temperature of between 120° C.-130° C. n-BuOAc (6000 mL) and water (200 mL) were collected by distillation at atmosphere. The mixture was then cooled to 100° C. and n-heptane (6000 mL) was added dropwise, maintaining the internal temperature between 90 and 98° C. The reaction mixture was cooled to room temperature overnight. The white suspension was filtered and the cake washed with n-heptane (4500 mL). The wet product was dried in a vacuum oven at 40° C. to give the aza-dione compound of formula III (1413.3 g, 95%) as an off-white solid.

### Example A3

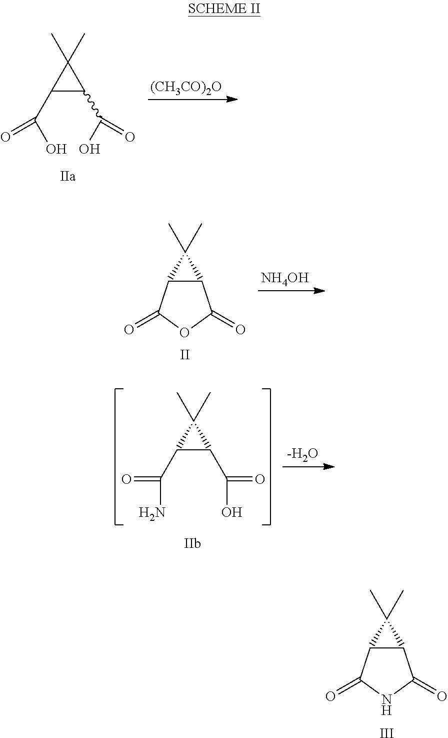

Preparation of Imide III from 3,3 dimethyl-cyclopropane-1,2-dicarboxylic acid (IIa) via Caronic anhydride II was carried out by slurrying 50 grams of cis/trans-3,3-dimethyl-1,2-cyclopropane dicarboxylic acid (IIa) in toluene (75 ml) and adding acetic anhydride (60 mL). After that conc. sulfuric acid (0.5 mL) was charged and the toluene was slowly distilled off. The reaction mixture was heated to about 190° C. while the remaining volatile compounds were collected by distillation. The reaction was cooled below 50° C. and THF (50 mL) was added. After cooling to about 0° C., ammonium hydroxide (32 mL, about 14.8N) was slowly charged while maintaining the temperature below 15° C. The mixture was then slowly heated to 110° C. while distilling off the THF. The reaction was further heated in stages to 180° C. After cooling and addition of THF (15 mL) the reaction was reheated to 140° C. while collecting the solvent by distillation. The mixture was cooled and THF (15 mL) and n-heptane (30 mL) were added. Distillation of solvent followed by cooling gave the crystalline imide III (Yield: 85%).

### Example A4

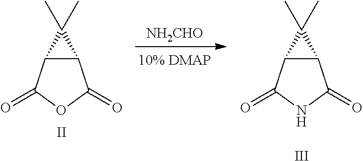

Into a three-necked, round bottom flask equipped with a temperature probe, condenser, and mechanical stirrer was placed 25.0 g of the compound of formula II (caronic anhydride). To the flask was added 9.37 mL of formamide (10.61 g, 0.424 equivalents based on anhydride) followed by 2.43 g of 4-N,N-dimethyl aminopyridine (DMAP, 0.1 equivalents). The vessel was purged with nitrogen and the reaction mixture heated to 145° C. with agitation, heating was continued for 2.5 hours. After proton NMR measurements indicated that the anhydride was completely consumed, the solution was cooled to 90° C. and the vessel was charged with 50 ml of xylenes (2 volumes). The reaction mixture was then heated to 145° C. with agitation. Heating was continued for 2.5 hours while operating the Dean-Stark condenser collecting a water/formamide azeotrope. After removal of excess formamide from the reaction mixture and conversion of all intermediates, the reaction mixture was cooled to 80° C. The reaction flask was then charged with 18.75 ml of heptanes (0.75 volumes) and the reaction mixture temperature was maintained at 80° C. After the addition of heptanes was complete, the reaction mixture was cooled over 2 hours to 0° C. and maintained in at a temperature of from 0° C. to 5° C. for 30 minutes with agitation. At the end of The reaction mixture was maintained in this temperature range with agitation for 30 minutes during which time a precipitate formed. The solids were collected by filtration and washed with two 50 mL aliquots of cold heptanes, and dried in a vacuum oven for 24 hours at 50° C.

### Alternative Procedure—Preparation of Benzyl Imide and Subsequent Reduction to Imide:

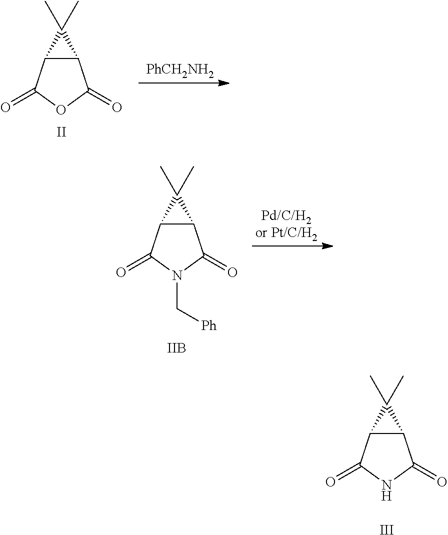

To a flask were charged 51.32 g of II (0.37 mol, 1 eq.) and 50 mL TBME. While stirring, the mixture was cooled to between 0 and 10° C. 40.0 mL of benzylamine (39.24 g, 0.37 mol, 1 eq) was added dropwise over approximately 30 minutes. After the addition was complete, the TBME was removed by distillation at between 60 and 70° C. and the mixture was gradually heated to an internal temperature between 170 and 180° C. The solution was maintained between 170 and 180° C. for approximately 3 to 5 hours to complete the cyclization. The resulting solution was cooled to between 60 and 70° C., and 100 mL of a solution of 5% water in isopropanol was added and the mixture was cooled to room temperature. After cooling further to between 0 and 10° C., the product was isolated by filtration, rinsed with clean, cold isopropanol, and dried in a vacuum oven to afford 70.99 g of the benzyl imide, IIB, (85%). ¹H NMR (CDCl₃) □ 7.39 (m, 2H); 7.28 (m, 3H); 4.53 (s, 2H); 2.31 (s, 2H); 1.20 (s, 3H); 1.01 (s, 3H). This product can be deprotected using conventional hydrogenolysis conditions (H₂, Pd/C) to afford III.

### Example 2

### Preparation of 6,6-Dimethyl-3-aza-bicyclo[3.1.0]hexane (IV)

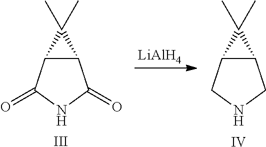

A THF solution of LiAlH₄ (500 mL, 2.4 M, 1.2 mol, 1.67 eq.) was charged into a 3-neck flask fitted with an N₂ inlet. The contents of the flask were warmed to 40° C. while being purged with nitrogen. 100 g of III (0.72 mol, 1 eq.) and 400 mL of THF were added to a second flask and stirred until a clear solution was formed. The solution containing III in the second 3-necked flask containing was then added over an approximately 0.5 to 1 hour period to the reaction mixture containing LiAlH₄ in the first 3-neck flask while allowing the temperature to rise to approximately 70° C. (reflux). The second flask was rinsed with 100 mL of THF, which was added to the reaction mixture to ensure complete transfer of III. Upon completion of the addition of the solution, the reaction mixture was maintained at reflux temperature and stirred until the reaction was complete (approximately 3 hours).

To a 3-necked flask fitted with a nitrogen inlet were charged 674 g of potassium sodium tartrate tetrahydrate (2.39 mol, 3.32 eq.) and 191 g of sodium hydroxide (4.78 mol, 6.64 eq.), 800 mL of H₂O and 300 mL TBME. The mixture was agitated between 15 and 25° C. for approximately 1 hour, or until all of the solids had dissolved. The reaction mixture was transferred via cannula to the biphasic quench mixture over approximately 10 to 20 minutes. The reaction flask was rinsed with 30 mL TBME which was also transferred via cannula to the quench flask. The biphasic mixture was agitated for an additional 15 to 30 minutes, and the layers were split at 40° C. The aqueous layer was extracted twice with 100 mL TBME. The combined organic layers were fractionally distilled to yield IV as a colorless liquid (64.5 g, 88%). ¹H NMR (CDCl₃, 400 MHz): δ 3.07 (m, 2H), 2.89 (d, 2H, J=11.6 Hz), 1.56 (br s, 1H), 1.25 (m, 2H), 1.00 (s, 3H), 0.98 (s, 3H).

Alternatively, compound IV in TBME solution from above was converted to its corresponding hydrochloric acid salt. First, the TBME was removed by distillation. Second a 18.6 g aliquot of the concentrated solution containing compound IV was taken and charged to a 500 mL, 3-neck flask equipped with mechanical stirrer, an N₂ line, a glass tube fixed through a 24-40 septa and an adapter to a 3N NaOH bubbler. The solution was cooled to −20° C. and held between −20 and −23° C. and gaseous HCl was bubbled through the solution while stirring for 10 minutes. A white precipitate was immediately apparent. The reaction was monitored by NMR and additional gaseous HCl was bubbled if necessary. The precipitate was filtered under a blanket of N₂ and washed with chilled heptanes (−60° C., 40 mL) under N₂ to give, after drying, 13.9 g, (70%) the IV*HCl salt. ¹H NMR (CDCl₃, 400 MHz): δ 7.90 (BS, 1H), 3.55 (d, J=16.4, 2H), 3.15 (d, J=16.4, 2H), 1.60 (m, 2H), 1.10 (s, 3H), 1.02 (s, 3H).

### Example 3

### Preparation of 6,6-dimethyl-3-aza-bicyclo[3.1.0]hex-2-ene (V) from amine (IV) via Chloroamine (IVa)

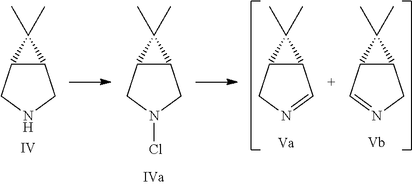

Into a 2 L round bottom flask equipped with a stirrer was charged 13% NaOCl (592 g, 1.15 eq.) and 250 ml of MTBE. Agitation was initiated and 100 g of the compound of Formula IV prepared in accordance with Example 2 above was slowly added while maintaining the reaction mixture at a temperature between 10° C. and 30° C. While maintaining the temperature the batch was agitated for 1 hr and sampled until GC indicated a conversion of greater than 99%. Additional MTBE (200 mL) was added to the reaction mixture and the batch was agitated while maintaining the temperature between 20° C. and 30° C. for 20 min and then the agitation was stopped and batch left to settle. The aqueous layer was split away. Into a second 2 L round bottom flask equipped with a stirring apparatus and a heater was placed 58 mL (720 g) of a 25% wt. aqueous NaOH solution NaOH, 25 g of a 40% wt. aqueous Bu₄NOH solution, and 50 mL of MeOH. The organic layer containing chloramine IVa obtained previously (50 ml) was slowly charged into the second reaction vessel, with agitation, while maintaining the reaction mixture at ambient temperature, about 20° C. to 25° C. The batch was heated over 20-30 minutes and the reaction mixture maintained at a temperature of between 50° C. and 55° C. during the reaction period. The reaction mixture was agitated for 8 hr whereupon GC indicated that greater than 99% of the chloroamine had converted to pyrrole. The reaction mixture was cooled to ambient temperature, about 20° C. to 25° C., agitation was stopped, and batch was settled. The aqueous layer was removed and the organic layer was washed with 20% brine twice (400 ml then 200 ml) to remove Bu4NOH (monitored by GC <0.3% Bu₄NOH). Removal of organic solvent under vacuum provided pure product (90% yield, >98% purity). ¹H NMR (CDCl₃, 400 MHz) δ 7.37 (m, 1H), 3.85 (m, 1H), 3.60 (m, 1H), 2.14 (m, 1H), 1.68 (m, 1H), 1.10 (s, 3H), 0.76 (s, 3H).

### Example 3A

### Preparation of Imine (V) Using Chiral PTC (Phase Transfer Catalyst)

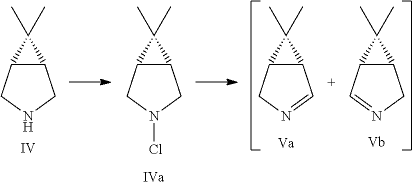

Into a 2 L round bottom flask equipped with a stirrer was charged 13% NaOCl (592 g, 1.15 eq.) and 250 ml of MTBE. Agitation was initiated and 100 g of the compound of Formula IV prepared in accordance with Example 2 above was slowly added while maintaining the reaction mixture at a temperature between 10° C. and 30° C. While maintaining the temperature the batch was agitated for 1 hr and sampled until GC indicated a conversion of greater than 99%. Additional MTBE (200 mL) was added to the reaction mixture and the batch was agitated while maintaining the temperature between 20° C. and 30° C. for 20 min and then the agitation was stopped and batch left to settle. The aqueous layer was split away. Into a second 2 L round bottom flask equipped with a stirring apparatus and a heater was placed 58 mL (72 g) of a 25% wt. aqueous NaOH solution NaOH, (8S,9R)-(−)-N-benzyl cinchonidinium chloride (19 g), and 50 mL of MeOH. The organic layer containing chloramine IVa obtained previously (50 ml) was slowly charged into the second reaction vessel, with agitation, while maintaining the reaction mixture at ambient temperature, about 20° C. to 25° C. The reaction mixture was agitated until GC indicated that greater than 92% of the chloroamine had converted to pyrrole. The reaction mixture was cooled to ambient temperature, about 20° C. to 25° C., agitation was stopped, and batch was settled. The aqueous layer was removed and the organic layer was washed with 20% brine twice (400 ml then 200 ml) to remove chiral PTC catalyst. Removal of organic solvent under vacuum provided pure product (80% yield, >98% purity, 20% ee), thus the reaction mixture was found to contain about 60 mole % of the compound of Formula Va and 40 mole % of the compound of Formula Vb. ¹H NMR (CDCl₃, 400 MHz) δ 7.37 (m, 1H), 3.85 (m, 1H), 3.60 (m, 1H), 2.14 (m, 1H), 1.68 (m, 1H), 1.10 (s, 3H), 0.76 (s, 3H).

### Comparative Example

### Preparation of Imine (V) Using Silver Mediated Persulfate Oxidation

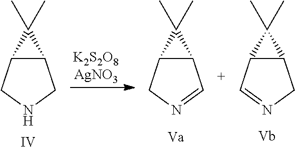

To a flask were charged 39.6 g of NaOH (0.99 mol, 2.2 eq.) and 158.1 g of K₂S₂O₈, 750 mL of water and 80 mL of acetonitrile at −5° C. 50 g of IV (0.45 mol, 1.0 eq) were added and the reaction mixture was again cooled to −5° C. 20 mL of aqueous AgNO₃ (2.29 g, 0.0135 mol, 0.03 eq) were added over 1-2 hours while maintaining the reaction temperature between −5 and 0° C. The reaction mixture was warmed to 0 to 2° C. and the reaction was allowed to proceed to completion. Upon completion, the mixture was warmed to room temperature and diluted with 360 mL TBME. The layers were separated, and the aqueous layer was extracted with TBME. The combined organic layers were dried over anhydrous Na₂SO₄ and filtered. The solution was purified by fractional distillation to yield V as a colorless oil which solidified upon standing to form a white crystalline solid racemic mixture of the compounds of the Formulae Va and Vb, (65-75 mole % yield based on starting compound of Formula IV, purity 92% via HPLC). ¹H NMR (CDCl₃) δ 7.30 (t, J=2.2 Hz, 1H), 3.80 (ddd, J=6.8, 1.4, 0.6 Hz, 1H), 3.49 (dd, J=4.7, 2.8 Hz, 1H), 2.06 (dd, J=6.0, 1.7 Hz, 1H), 1.61 (dd, J=6.6, 1.8 Hz, 1H), 1.03 (s, 3H), 0.68 (s, 3H).

While the present invention has been described with and in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A process for the provision of a mixture of compounds of the formulae (Va) and (Vb). 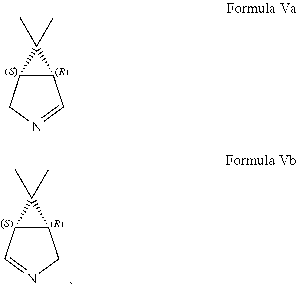
the process comprising:
(a) treating a pyrrolidine compound of Formula IV, 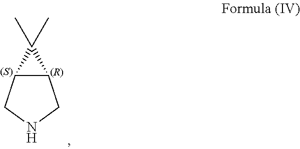
with a chlorinating agent to form the compound of Formula IVa, 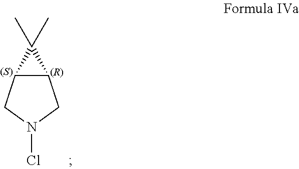
and
(b) dehydrochlorinating the chloroamine obtained in Step “a” to produce the compounds of Formulae VIa and VIb.

2. The process according to claim 1, wherein the chlorinating agent is selected from sodium hypochlorite and N-chlorosuccinimide.

3. The process according to claim 2, wherein the reaction is carried out in a reaction mixture comprising a phase transfer catalyst selected from (8S,9R)-(−)-N-benzyl cinchonidium chloride, tetraoctyl ammonium chloride, tetrabutyl ammonium chloride, and tetrabutyl ammonium hydroxide.

4. The process according to claim 3, wherein the reaction mixture further comprises a promoter.

5. The process according to claim 4, wherein the promoter is methanol.

6. The process according to claim 1, wherein the pyrrolidine compound of Formula IV is prepared by a process comprising:
(a) providing the compound of Formula IIB 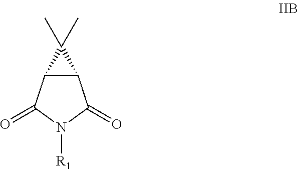
wherein R₁ is aralkyl, substituted aralkyl, or alkenyl; and
(b) reducing the carbonyl groups and hydrogenating the pyrrolidine nitrogen in either order to provide the compound of Formula IV.

7. The process according to claim 6, wherein R₁ is benzyl or allyl.

8. The process of claim 1 wherein the chlorination reagent is substituted for a reagent selected from an iodination reagent and a bromination reagent, thereby providing an intermediate haloamine which is converted to compounds of Formula VIa and VIb in step 2 by, respectively, dehydroiodination and dehydrobromination.
